(19) United States
(12) Patent Application Publication
(10) Pub. No.: US 2014/0228740 A1
(43) Pub. Date: Aug. 14, 2014

(54) METHOD OF PRODUCING SUBSTANCES WITH SUPERSATURATED GAS, TRANSDERMAL DELIVERY THEREOF, AND USES THEREOF

(71) Applicant: Norbert Kiss, La Crescenta, CA (US)

(72) Inventor: Norbert Kiss, La Crescenta, CA (US)

(73) Assignee: INVISIDERM, LLC, Pasadena, CA (US)

(21) Appl. No.: 14/112,895

(22) PCT Filed: Apr. 19, 2011
(86) PCT No.: PCT/US11/33060

§ 371 Date: Dec. 9, 2013

(51) Int. Cl. A61M 35/00; A61M 37/00

(52) U.S. Cl. CPC A61M 35/00; A61M 37/00; A61M 2202/02
USPC 604/24

(57) ABSTRACT

The present specification disclosed a noninvasive transdermal delivery device that relates generally to a handheld mechanical apparatus for noninvasive transdermal administration of gas, small to large water-soluble (hydrophilic) pharmaceutical agents, vitamins, and other therapeutic agents. Components of such delivery devices, methods of producing a substance comprising a supersaturated amount of a dissolved gas, as well as, methods of administering a therapeutic agent using such delivery devices and methods of treating a disease or condition using such delivery devices are also disclosed.

### INTRODUCTION

Delivery of an agent through the skin to achieve a therapeutic effect is commonly known as transdermal drug delivery. Transdermal drug delivery systems are dosage forms that facilitate transport of a therapeutic agent to viable epidermal and or dermal tissues of the skin for local therapeutic effect as well as systemically via blood circulation. Transdermal delivery of a therapeutic agent provides several advantages over injectable and oral routes. For example, transdermal delivery of a therapeutic agent increases bioavailabiltity of the agent by avoiding gastrointestinal absorption and hepatic first pass metabolism, enhances therapeutic efficiency of the agent by providing controlled, constant administration of the agent, maintains a steady plasma level of the agent by providing continuous administration of the agent, reduces pharmacological dosing due better absorption of the agent, and provides better overall treatment value through greater administration flexibility and increase patient compliance. Disadvantages of transdermal delivery include, e.g., difficulty in administering therapeutic agents with a molecular weight greater than 500 Daltons or use of therapeutic agents with a very low or high partition coefficient.

A transdermal drug delivery system may be of an active or a passive design. Common dosage forms of a passive transdermal drug delivery system include, e.g., ointments, creams, gels, and transdermal patches. Passive systems require careful selection of a base and addition of penetration enhancers and are applied the skin surface to deliver a specific dose of agent into the blood stream. Because of the impervious nature of the skin, passive transdermal drug delivery systems have typically been used with lipophilic therapeutic agents.

An active transdermal drug delivery system uses mechanical energy to increase therapeutic agent transport across the skin by either altering the skin barrier (primarily the stratum corneum) or increasing the agent's energy. Such active systems include, e.g., microneedles and microdermabrasion which puncture or otherwise physically disrupt the stratum corneum, photochemical waves which use chemicals to alter the stratum corneum, iontophoresis which uses low voltage electrical current to drive charged agents through the skin, electroporation and reverse electrporation which use short high voltage electrical pulses to create transient aqueous pores in the skin, sonophoresis which uses low frequency ultrasonic energy to disrupt the stratum corneum, thermal ablation which uses heat to make the skin more permeable and to increase the agent's energy, and magnetophoresis which uses magnetic energy to increase drug flux across the skin.

There are two important layers in skin: the dermis and the epidermis. The outermost layer, the epidermis, is approximately 100 to 150 micrometers thick, has no blood flow and includes a layer within it known as the stratum corneum. This layer is important to transdermal delivery as its composition provides for water retention and foreign substance defense. Beneath the epidermis, the dermis contains a system of capillaries that transport blood throughout the body. If the drug is able to penetrate the stratum corneum, it can enter the blood stream. Although sweat ducts and hair follicles are also paths of entry into the blood system, these avenues have been considered rather insignificant. See, e.g., Aulton, Pharmaceutics: The Science of Dosage Form Design (2d edition, Churchill Livingston, Harcourt publishers, 2002).

The transdermal route has become one of the most successful and innovative focus's for research in drug delivery. Over 35 therapeutic agents have now been approved for sale in the U.S., and approximately 16 active ingredients have been approved for use globally. However, there is still a need for better ways to deliver a therapeutic agent by the transdermal route. For example, transdermal delivery systems on the market today are limited to small molecular weight drugs with very small daily dosages often companied by various patient discomforts. The present specification discloses a transdermal delivery system that uses a device to administer a vapor comprising liquid particles including a supersaturated amount of a dissolved therapeutic agent that enters the circulatory system via the sweat gland pore and duct system.

### SUMMARY

Thus, aspects of the present specification disclose a transdermal device. A transdermal delivery device disclosed herein comprising a housing and a vapor producing assembly, wherein the housing encloses the vapor producing assembly.

Other aspects of the present specification disclose a vapor producing assembly. A vapor producing assembly disclosed herein comprises a pressure-temperature regulator assembly and a fluid chamber assembly. A vapor producing assembly disclosed herein may optionally comprise a control switch assembly.

Yet other aspects of the present specification disclose a method of producing a substance comprising a supersaturated amount of dissolved gas. A method of producing a substance disclosed herein comprises the steps of placing a substance as disclosed herein into an air-tight container; and exposing the substance to gas, wherein upon exposure, the gas dissolves into the substance in an amount greater than the substance could dissolve at 25° C. and 1 atm. The gas may be carbon dioxide. The resulting substance supersaturated with the dissolved gas can then be administered to an individual to treat a condition as disclosed herein. In another aspect, the present specification disclose a use of a substance comprising a supersaturated amount of dissolved gas to manufacture a medicament. Such a medicament can then be administered to an individual to treat a condition as disclosed herein.

Still other aspects of the present specification disclose a method of transdermally administering a therapeutically effective amount of therapeutic agent. A method of transdermal administration disclosed herein comprises the step of administering a substance comprising a supersaturated amount of dissolved gas to an individual using a transdermal delivery device disclosed herein. In another aspect, a method of transdermal administration disclosed herein comprises the step of administering a substance comprising a supersaturated amount of dissolved gas and a therapeutic agent to an individual using a transdermal delivery device disclosed herein. Administration of the gas and/or the therapeutic agent typically treats a symptom associate with a condition. In an aspect, the present specification disclose a use of a substance including a supersaturated amount of dissolved gas to treat a condition using a transdermal device disclosed herein.

Further aspects of the present specification disclose a method of treating a condition using a transdermal device disclosed herein. A method of treating a condition disclosed herein comprises the step of administering a composition comprising a substance including a supersaturated amount of dissolved gas and a therapeutic agent using a transdermal delivery device as disclosed herein to a body part of the individual suffering from a condition, wherein the administration of the composition reduces a symptom associated with condition. A substance may be a liquid aerosol, foam, emulsion, gel, sol, or other substance that can become supersaturated with an amount of dissolved gas. A condition includes an imperfection, a defect, a disease, and/or a disorder for which relief is sought by the individual suffering from the condition. In an aspect, the present specification disclose a use of a substance including a supersaturated amount of dissolved gas to treat a condition. A transdermal device disclosed herein may be used to administer the substance.

### BREIF DESCRIPTION OF THE DRAWINGS

The figures are exemplary of different embodiments of the subject matter disclosed herein. Each illustrated embodiment is not intended to limit the scope of the subject matter disclosed herein, but rather, be exemplary to the scope and spirit of it. Like components in the figures share identical numbering.

FIG. 1 illustrates a cross-section view of an exemplary transdermal delivery device.

FIG. 2 illustrates a cross-section view of an exemplary housing.

FIG. 3 illustrates an exemplary vapor producing assembly. FIG. 3A shows a perspective view. FIG. 3B shows a cross-section view.

FIG. 4 illustrates a cross-section view of an exemplary pressure-temperature regulator assembly similar to that shown in FIG. 3, comprising a non-threaded lance housing.

FIG. 5 illustrates a cross-section view of an exemplary pressure-temperature regulator assembly similar to that shown in FIG. 3, comprising an adjustable height plunger and non-threaded lance housing.

FIG. 6 illustrates a cross-section view of an exemplary pressure-temperature regulator assembly similar to that shown in FIG. 3, comprising an adjustable height plunger and a threaded lance housing

FIG. 7 illustrates a cross-section view of an exemplary pressure-temperature regulator assembly similar to that shown in FIG. 3, comprising a threaded lance housing

FIG. 8 illustrates a cross-section view of an exemplary control switch assembly.

FIG. 9 illustrates a cross-section view of an exemplary fluid chamber assembly.

FIG. 10 illustrates a cross-section view of an exemplary transdermal device.

### DETAILED DESCRIPTION

The average diameter of most human sweat glands offers adequate space for most drug molecules to pass through. According to various studies, the average density of sweat pores varies greatly with the individual and body site. The palmer surfaces, palms and finger, and the plantar surfaces, soles of the feet and the toes have an average of 2,700 pores per square inch of ridge friction skin surface. This compares to approximately 400 pores per square inch of the balance of the body's skin surface. The total number of sweat pores distributed over the entire body has been estimated at from 1.6 to four million. The size of the sweat gland has been found to vary as much as fivefold between individuals but on average the pore size in the human skin is 50 microns. The dimension of the coil leading down from the opening in the epidermis is about two to five mm long and about 60 to 80 microns in diameter, with the duct having a slightly smaller diameter.

The present specification discloses lightweight, hand-held mechanical devices designed to transdermally administer therapeutic agents to an individual. The devices produce a vapor comprising a supersaturated amount of a dissolved gas that is non-invasively delivery through the skin via the pore and duct systems contained within the skin, such as, e.g., the sweat gland pore and duct system. In general operation, a removable cartridge containing a compressed gas like carbon dioxide is attached to a port of a pressure-temperature regulator assembly. The regulator assembly reduces the pressure, and thus the temperature and speed of gas flow, to a preset level useful to the purposes disclosed herein. In the case where the gas is the therapeutic agent, this low pressure, ambient temperature gas is then passed to a fluid chamber assembly containing a liquid, such as, e.g., water, a physiologically buffered solution, or other suitable liquid, where it is dissolved into the liquid producing a liquid supersaturated with the gas. This therapeutic gas is then administered to an individual by vaporizing the supersaturated liquid and applying the vapor to a skin surface where the liquid particles including a supersaturated amount of dissolved therapeutic agent enters into the body via skin pores. In the case where the therapeutic agent is not the gas, this low pressure, ambient temperature gas is passed to a fluid chamber assembly containing a liquid and the therapeutic agent where the gas is dissolved into the liquid producing a therapeutic liquid including a supersaturated amount of dissolved gas. This therapeutic agent is then transdermally administered to an individual as a vapor.

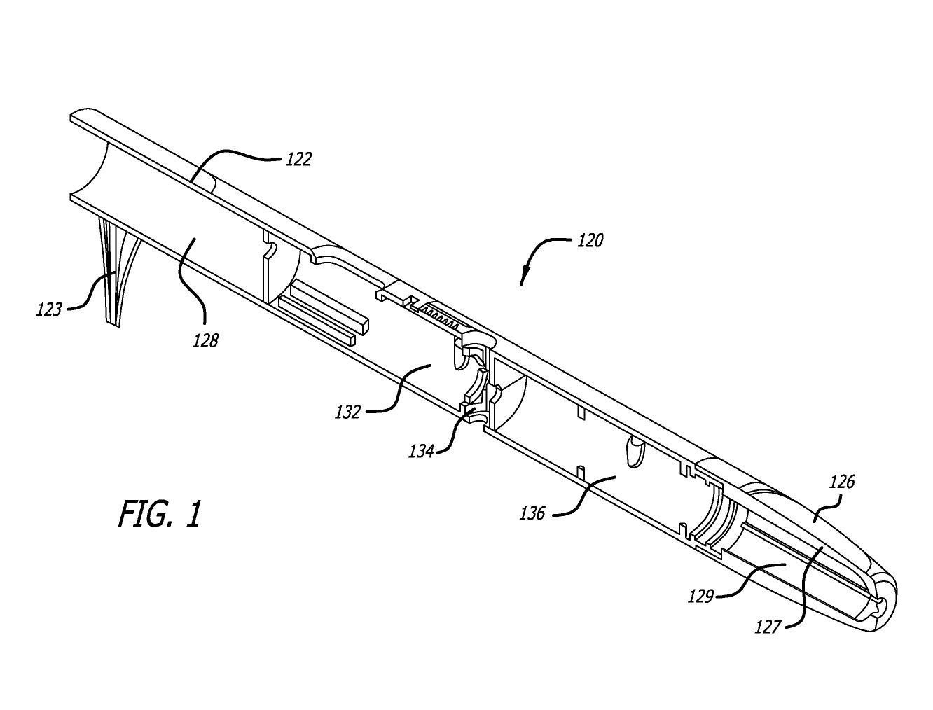

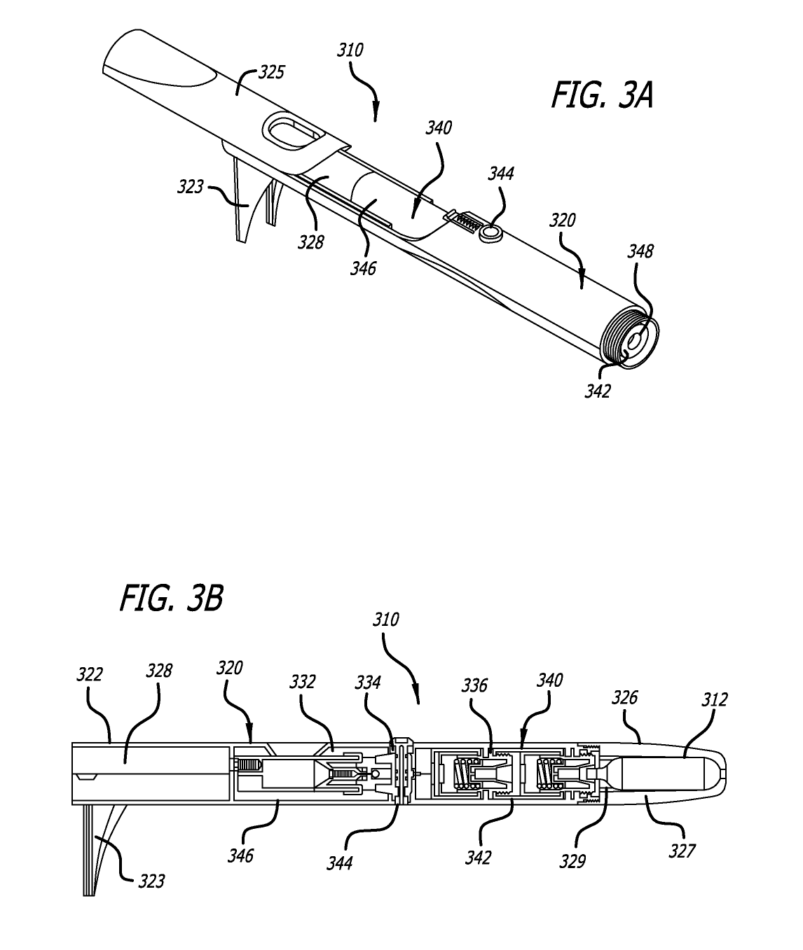

Aspects of the present specification disclose, in part, a transdermal delivery device. A transdermal delivery device disclosed herein comprising a housing (see, e.g., housing 120 of FIG.1) and a vapor producing assembly (see, e.g., 340 of FIG. 3), wherein the housing encloses the vapor producing assembly. Such a device is designed to be a lightweight, hand-held portable device that provides a practical and comfortable feel for the user during operation of the device. The overall shape of the transdermal delivery device disclosed herein is generally cylindrical in shape, although other geometries can be used. In one embodiment, a transdermal delivery device disclosed herein has a length of less than about 20 inches long, less than about 18 inches long, less than about 16 inches long, less than about 14 inches long, or less than about 12 inches long, and a width of less than 2 inches, less than 1.5 inches, less than 1 inches, or less than 0.5 inches. In an aspect of this embodiment, a transdermal delivery device disclosed herein is less than about 16 inches in length and about 1.5 inches in width.

A housing disclosed herein comprises an external body shell (see, e.g., external body shell 122 of FIG. 1), one or more internal compartments and a cartridge retaining container detachably engaged to the external body shell (see, e.g., cartridge retaining container 126 of FIG. 1). An external shell disclosed herein can be made of any durable material that provides for durability, safety, and portability, including a metal or metal alloy, a high-strength plastic, or a composite material. The shape of the external shell is designed to contain a vapor producing assembly disclosed herein and provide a practical and comfortable fell when held in the hand of the user and during operation of the device.

A housing disclosed herein may optionally comprise a fluid chamber assembly access covering detachably engaged with the external body shell. A fluid chamber assembly access covering disclosed herein is designed to provide access to a fluid chamber assembly disclosed herein. A fluid chamber assembly access covering disclosed herein is designed to be detached from the external body shell of the transdermal delivery device. Such detachment allows a user to, e.g., remove a fluid chamber assembly, or component thereof, as well as reattach a fluid chamber assembly, or component thereof. In one embodiment, a fluid chamber assembly access covering disclosed herein is designed to be completely removed from the housing of the transdermal delivery device in order to allow access as disclosed herein. In another embodiment, a fluid chamber assembly access covering disclosed herein is designed to achieve access to the fluid chamber assembly as disclosed herein, but still remain attached to the external body shell of the housing. As a non-limiting example, a fluid chamber assembly access covering may include a threaded portion that can be screwed onto or off of a threaded portion of the external body shell. In such an arrangement, the asses covering can be completely removed from the housing. As another non-limiting example, a fluid chamber assembly access covering includes a track and grove arrangement with the external body shell allowing the access covering to slide back and forth from an open to close position. Such an arrangement can be designed to allow complete removal of the access covering or include a stop that prevents complete removal, but provides access as disclosed herein. As yet another non-limiting example, a fluid chamber assembly access covering including a hinge assembly with the external body shell that allows the access covering to be swung open or closed. In such an arrangement, the access covering typically remained attached to the housing. Other arrangements to allow asses as disclosed herein are known in the art.

The one or more internal compartments disclosed herein are designed to correctly hold a vapor producing assembly, or components thereof, in a manner that ensures proper operation of the transdermal delivery device. An internal compartment includes, without limitation, an open-ended delivery outlet (see, e.g., open-ended delivery outlet 128 of FIG. 1) and a vapor producing assembly compartment (see, e.g., vapor producing assembly compartment 130 of FIG. 1). A vapor producing assembly compartment may be further subdivided and include a fluid chamber assembly compartment (see, e.g., fluid chamber assembly compartment 132 of FIG. 1), a control switch assembly compartment (see, e.g., control switch assembly compartment 134 of FIG. 1), and/or a pressure-temperature regulator assembly compartment (see, e.g., pressure-temperature regulator assembly compartment 136 of FIG. 1). Such compartments may include internal struts that enhance structural integrity of the device and/or footings that ensure proper placement and function of the vapor producing assembly disclosed herein, or component part thereof.

A cartridge retaining container disclosed herein comprises an external covering shell (see, e.g., external covering shell 127 of FIG. 1) and an internal cartridge compartment (see, e.g., internal cartridge compartment 129 of FIG. 1). A cartridge retaining container disclosed herein is designed to correctly position, mount, and secure a compressed gas cartridge to a lance housing of a pressure-temperature regulator assembly during properly operation of the transdermal delivery device.

A cartridge retaining container disclosed herein is designed to detachable engage the external body shell of the housing. In one embodiment, a cartridge retaining container disclosed herein is designed to be completely removed from the housing of the transdermal delivery device in order to achieve an unengaged position as disclosed herein. In another embodiment, a cartridge retaining container disclosed herein is designed to achieve an unengaged position as disclosed herein, but still remain attached to the housing. As a non-limiting example, a cartridge retaining container may include a threaded portion that can be screwed onto or off of a threaded portion of the external body shell. In such an arrangement, the cartridge retaining container can be completely removed from the housing where a compressed gas cartridge is inserted into an internal cartridge compartment. The cartridge retaining container is then screwed back onto the housing in a manner that allows properly insertion of the cartridge into the device. As another non-limiting example, a cartridge retaining container including a hinge assembly with the external body shell that allows the cartridge retaining container to positioned in a manner that allows a compressed gas cartridge to be properly inserted into the device. In such an arrangement, the cartridge retaining container typically remained attached to the housing. Other arrangements to allow proper cartridge insertion and cartridge retaining container attachment as disclosed herein are known in the art.

A cartridge retaining container disclosed herein is designed to be detachably engaged with the external body shell of the transdermal delivery device. This is achieved in that a cartridge retaining container disclosed herein can be in one of two operational positions. In an unengaged position (or detached or opened position), a cartridge retaining container disclosed herein allows a compressed gas cartridge to be placed in the internal cartridge compartment of the cartridge retaining container, reveals a lance housing present on a pressure-temperature regulator assembly disclosed herein for a compressed gas cartridge, and/or both. In an engaged position (or attached or closed position), a cartridge retaining container disclosed herein is designed to position, mount, and secure a compressed gas cartridge to a lance housing of a pressure-temperature regulator assembly in a manner that releases the compressed gas from the cartridge and channels the released gas into the pressure-temperature regulator assembly.

A housing disclosed herein may optionally comprise a leg stand attached to the external body shell (see, e.g., leg stand 123 of FIG. 1). A leg stand disclosed herein is typically located near the end where the fluid container assemble is located. A leg stand disclosed herein is designed to angle a fluid container assemble to provide a tilt of no greater than 30° relative to a horizontal position of a transdermal delivery device in order to facilitate mixing of the gas and liquid.

Thus, in one embodiment, a housing as disclosed herein comprises an external body shell, an open-ended delivery outlet, a vapor producing assembly compartment, and a cartridge retaining container detachably engaged with the external body shell, wherein the vapor producing assembly compartment intervenes between the open-ended delivery outlet and the cartridge retaining container. The open-ended delivery outlet is designed to receive a body part of an individual such as a finger, toe, or paw. Alternatively, the open-ended delivery outlet may simply be place on top, or in the vicinity of, a skin surface. The vapor producing assembly compartment itself can be subdivided into different compartments designed to contain component parts of the vapor producing assembly disclosed herein.

In another embodiment, a housing disclosed herein comprises an external body shell, an open-ended delivery outlet, a vapor producing assembly compartment comprising a fluid chamber assembly compartment and a pressure-temperature regulator assembly compartment, and a cartridge retaining container detachably engaged with the external body shell, wherein the linear arrangement of the interior compartments is the open-ended delivery outlet next to the fluid chamber assembly compartment which is next to the pressure-temperature regulator assembly compartment.

In yet another embodiment, as shown in FIG. 1, housing 120 comprises external body shell 122, leg stand 123, open-ended delivery outlet 128, vapor producing assembly compartment 130 comprising fluid chamber assembly compartment 132, control switch assembly compartment 134, pressure-temperature regulator assembly compartment 136 and cartridge retaining container 126 detachably engaged with external body shell, and comprises an external covering shell 127, an internal cartridge compartment 129.

The transdermal device may optionally comprise a compressed gas cartridge. A compressed gas cartridge disclosed herein is typically of a size sufficient to contain enough gas under pressure to produce a volume of liquid supersaturated with dissolved gas sufficient to produce a vapor that provides a therapeutic effect with one dose. A compressed gas cartridge disclosed herein typically contains a gas having a pressure exceeding 40 psi (about 275 kPa) at 21.1° C., or regardless of the pressure at 21.1° C., having a pressure exceeding 104 psi (about 717 kPa) at 54.4° C., or any liquid having an absolute vapor pressure exceeding 40 psi (about 275 kPa) at 37.8° C. For example, a compressed gas cartridge containing 16 g, 8 g, or 1.3 g of a food or medical grade gas under a pressure of about 400 kPa about 58 psi), about 600 kPa about 87 psi), about 800 kPa (about 116 psi), or about 1000 kPa (about 145 psi) at 21.1° C. In one embodiment, a compressed gas cartridge containing 16 g of food or medical grade carbon dioxide under about 800 kPa of pressure at 21.1° C. The compressed gas cartridge may be of a disposable design. Such a disposable compressed gas cartridge typically includes a permeation seal that can be pierced to release the gas. For example, as disclosed herewithin, a lance from a pressure-temperature regulator assembly pierces the permeation seal of a compressed gas cartridge, thereby allowing release of compressed gas from the cartridge into the pressure-temperature regulator assembly in a manner that ensures proper operation of the transdermal delivery device. Non-limiting examples of a gas useful to operate the transdermal delivery device disclosed herein include a food or medical grade gas including a food or medical grade carbon dioxide, a food or medical grade oxygen, a food or medical grade helium, and a food or medical grade argon. A compressed gas cartridge disclosed herein can be threaded or non-threaded. A threaded compressed gas cartridge can be secured to a pressure-temperature regulator assembly without the aid of a cartridge retaining container as disclosed herein. A non-threaded compressed gas cartridge is secured to a pressure-temperature regulator assembly using a cartridge retaining container as disclosed herein. A compressed gas cartridge disclosed herein may be of a standard industry design, or may be of a custom design useful solely for the transdermal delivery device disclosed herein. In one embodiment, a threaded or non-treaded compressed gas cartridge comprises a body, an internal gas compartment, and a permeation seal. In another embodiment, a threaded or non-treaded compressed gas cartridge comprises a body, a neck, an internal gas compartment, and a permeation seal.

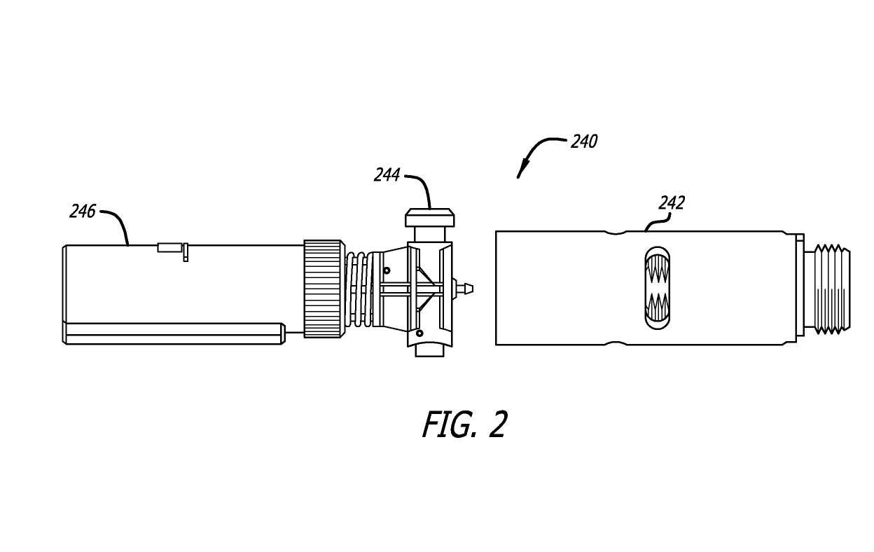

Aspects of the present specification disclose, in part, a vapor producing assembly. A vapor producing assembly disclosed herein comprises a pressure-temperature regulator assembly and a fluid chamber assembly. A vapor producing assembly disclosed herein may optionally comprise a control switch assembly. In one embodiment, a vapor producing assembly disclosed herein comprises a pressure-temperature regulator assembly and a fluid chamber assembly, but not a control switch assembly. In another embodiment, a vapor producing assembly disclosed herein comprises a pressure-temperature regulator assembly, a control switch assembly, and a fluid chamber assembly. In yet another embodiment, as shown in FIG. 2, a vapor producing assembly 240 comprises a pressure-temperature regulator assembly 242, a control switch assembly 244, and a fluid chamber assembly 246, and, optionally, a compressed gas cartridge 212.

In one embodiment, as shown in FIG. 3, transdermal delivery device 310 comprises a housing 320 with leg stand 323 and fluid chamber access covering 325. Fluid chamber assembly access covering 325 is opened to shown open-ended delivery outlet 328 and fluid chamber assembly 346. Fluid chamber assembly access covering has a track and grove arrangement with the external body shell allowing the access covering to slide back and forth. Cartridge retaining container is detached from transdermal delivery device 310 to expose pressure-temperature regulator assembly 342 and lance housing 348 for compressed gas cartridge. Cartridge retaining container (not shown) has a threaded portion that can be screwed onto or off of a threaded portion of the external body shell 320.

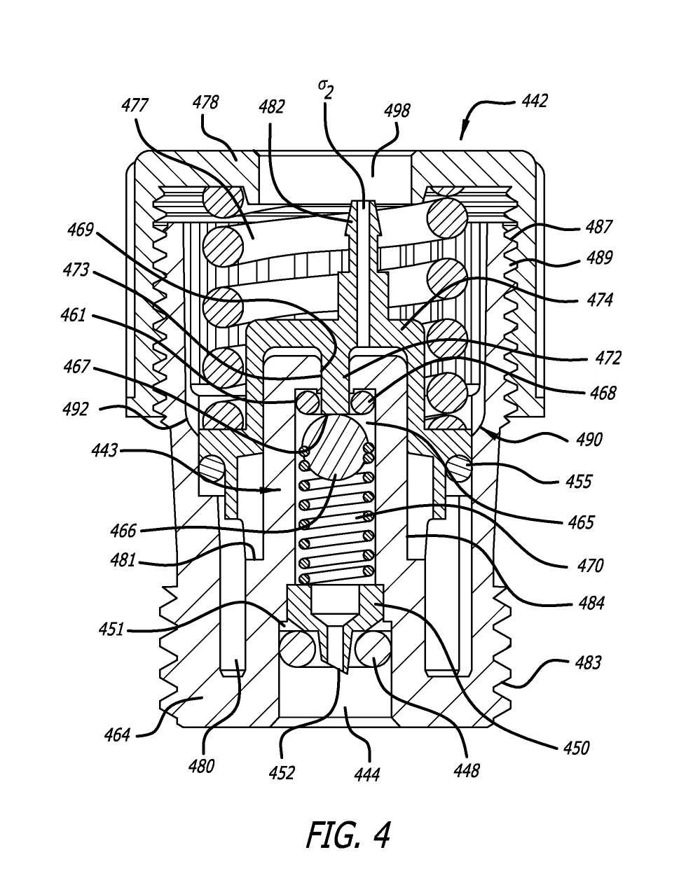

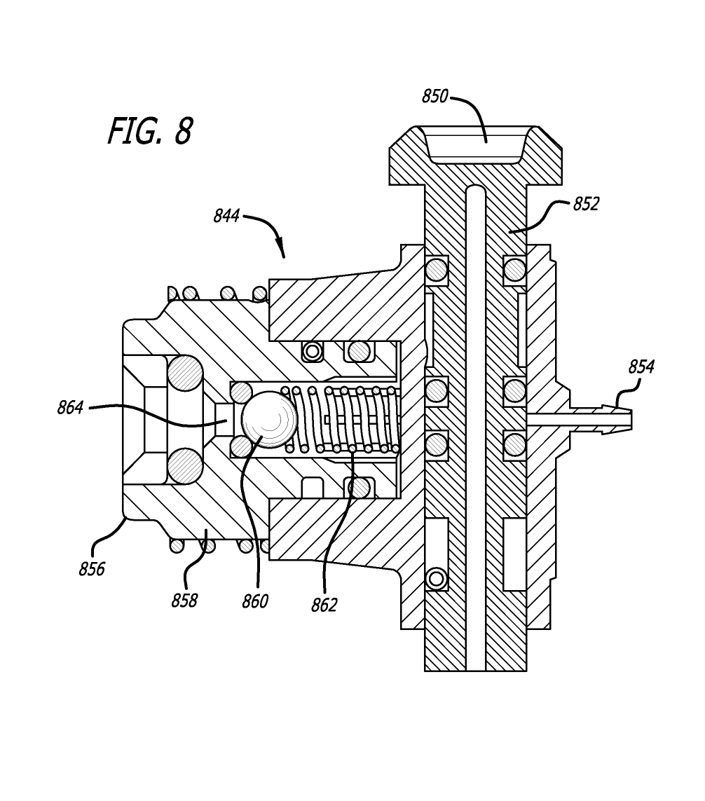

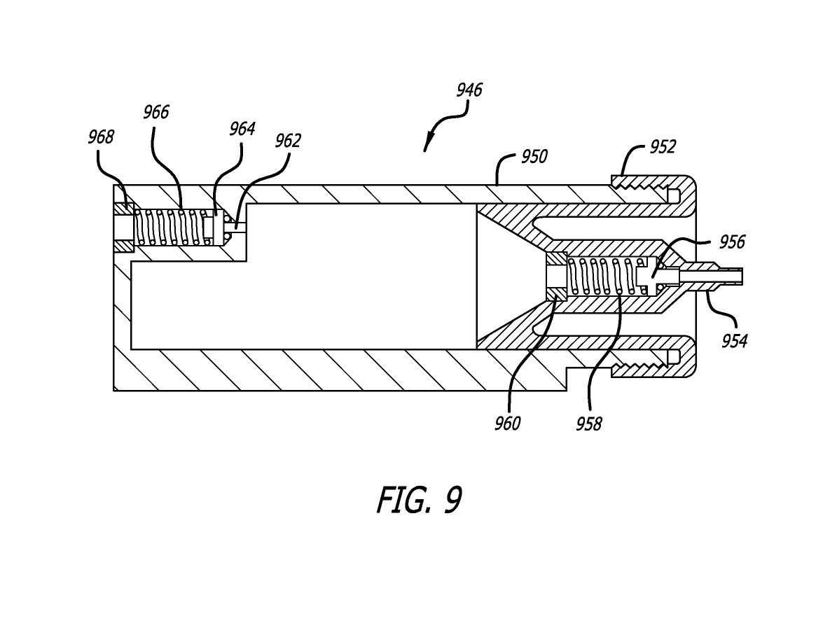

FIG. 3B is a cross-sectional view of transdermal delivery device 310 comprising housing 320 including external shell 322, open-ended delivery outlet 328, vapor producing assembly compartment 330 comprising fluid chamber assembly compartment 332, control switch assembly compartment 334, pressure-temperature regulator assembly compartment 336 and cartridge retaining container 326 removable engaged with the external body shell. Within housing 320 is vapor producing assembly 340 comprises pressure-temperature regulator assembly 342, control switch assembly 344, and fluid chamber assembly 346, and, optionally, compressed gas cartridge 312. Pressure-temperature regulator assembly 342 may be one of the pressure regulator assemblies described in FIGS. 4-7, or be one of a different design, but of similar function. Control switch assembly 344 may be the control switch assembly described in FIG. 8, or be one of a different design, but of similar function. Fluid chamber assembly 346 may be the fluid chamber assembly described in FIG. 9, or be one of a different design, but of similar function.

A pressure-temperature regulator assembly disclosed herein comprises at least one pressure regulator. A pressure-temperature regulator assembly disclosed herein is designed to reduce the pressure and speed as well as increase the temperature of a gas coming from a compressed gas cartridge so that when the gas enters into a fluid chamber assembly as disclosed here the gas will freeze the liquid, but instead dissolve into it. A pressure-temperature regulator assembly disclosed herein comprises a pressure regulator including a lance housing with lance, a regulator body, a piston, and an outlet port and can be made from metal, a metal alloy, high strength glass reinforced nylon, or other lightweight material that can withstand the high pressure and cold temperature exerted by the gas as it leaves the compressed gas cartridge. A pressure-temperature regulator assembly disclosed herein comprising two or more pressure regulators will further comprise an adaptor. The adaptor connects the pressure regulators to one another thereby creating a channel for the gas to flow through. For example, in a pressure-temperature regulator assembly comprising two pressure regulators, the adaptor connects the first pressure regulator to the second pressure regulator. As another non-limiting example, in a pressure-temperature regulator assembly comprising three pressure regulators, a first adaptor connects the first pressure regulator to the second pressure regulator, and a second adaptor connects the second pressure regulator to the third pressure regulator. Exemplary pressure regulators useful to operate the transdermal delivery device disclosed herein are described in, e.g., Hollars, Pressure Regulator adaptable to Compressed Gas Cartridge, U.S. Pat. No. 7,334,598, which is hereby incorporated by reference in its entirety for all that it discloses regarding pressure regulators.

In one embodiment, a pressure-temperature regulator assembly disclosed herein is set to reduce the pressure of a compressed gas to below about 40 psi (about 275 kPa) at 21.1° C. In aspects of this embodiment, a pressure-temperature regulator assembly is set to reduce the pressure of a compressed gas to, e.g., about 35 psi (about 241 kPa), about 30 psi (about 207 kPa), about 25 psi (about 172 kPa), about 20 psi (about 138 kPa), or about 15 psi (about 103 kPa). In other aspects of this embodiment, a pressure-temperature regulator assembly is set to reduce the pressure of a compressed gas to, e.g., below 40 psi (about 275 kPa), below 35 psi (about 241 kPa), below 30 psi (about 207 kPa), below 25 psi (about 172 kPa), below 20 psi (about 138 kPa), or below 15 psi (about 103 kPa). In yet other aspects of this embodiment, a pressure-temperature regulator assembly is set to reduce the pressure of a compressed gas to between, e.g., about 15 psi (about 103 kPa) to about 40 psi (about 275 kPa), about 15 psi (about 103 kPa) to about 35 psi (about 241 kPa), about 15 psi (about 103 kPa) to about 30 psi (about 207 kPa), about 15 psi (about 103 kPa) to about 25 psi (about 172 kPa), or about 15 psi (about 103 kPa) to about 20 psi (about 138 kPa).

In another embodiment, a pressure-temperature regulator assembly disclosed herein increases the temperature of a compressed gas so that when the gas is leaves from regulator assembly and enters a fluid chamber assembly, the gas will not freeze a liquid contained in the fluid chamber assembly. In aspects of this embodiment, a pressure-temperature regulator assembly increases the temperature of a compressed gas to, e.g., about 0° C., about 2° C., about 4° C., about 5° C., about 8° C., about 10° C., about 12° C., about 15° C., about 18° C., about 20° C., or about 22° C. In other aspects of this embodiment, a pressure-temperature regulator assembly increases the temperature of a compressed gas to, e.g., at least 0° C., at least 2° C., at least 5° C., at least 8° C., at least 10° C., at least 12° C., at least 15° C., at least 18° C., at least 20° C., or at least 22° C. In yet other aspects of this embodiment, a pressure-temperature regulator assembly increases the temperature of a compressed gas to between, e.g., about 0° C. to about 22° C., about 2° C. to about 22° C., about 4° C. to about 22° C., about 8° C. to about 22° C., about 12° C. to about 22° C., about 0° C. to about 18° C., about 2° C. to about 18° C., about 4° C. to about 18° C., about 8° C. to about 18° C., or about 12° C. to about 18° C.

In another embodiment, a pressure-temperature regulator assembly disclosed herein is set to reduce the pressure of a compressed gas to about 15 psi (about 103 kPa) to about 40 psi (about 275 kPa) and increase the temperature of the compressed gas to about 0° C. to about 22° C. In another embodiment, a pressure-temperature regulator assembly disclosed herein is set to reduce the pressure of a compressed gas to about 15 psi (about 103 kPa) to about 40 psi (about 275 kPa) and increase the temperature of the compressed gas to about 4° C. to about 22° C. In another embodiment, a pressure-temperature regulator assembly disclosed herein is set to reduce the pressure of a compressed gas to about 15 psi (about 103 kPa) to about 40 psi (about 275 kPa) and increase the temperature of the compressed gas to about 8° C. to about 22° C. In another embodiment, a pressure-temperature regulator assembly disclosed herein is set to reduce the pressure of a compressed gas to about 15 psi (about 103 kPa) to about 40 psi (about 275 kPa) and increase the temperature of the compressed gas to about 12° C. to about 22° C.

In another embodiment, a pressure-temperature regulator assembly disclosed herein is set to reduce the pressure of a compressed gas to below about 40 psi (about 275 kPa) and increase the temperature of the compressed gas to at least 0° C. In another embodiment, a pressure-temperature regulator assembly disclosed herein is set to reduce the pressure of a compressed gas to below about 40 psi (about 275 kPa) and increase the temperature of the compressed gas to at least 4° C. In another embodiment, a pressure-temperature regulator assembly disclosed herein is set to reduce the pressure of a compressed gas to below about 40 psi (about 275 kPa) and increase the temperature of the compressed gas to at least 8° C. In another embodiment, a pressure-temperature regulator assembly disclosed herein is set to reduce the pressure of a compressed gas to below about 40 psi (about 275 kPa) and increase the temperature of the compressed gas to at least 12° C.

In one embodiment, as shown in FIG. 4, pressure-temperature regulator assembly 442 comprises adjuster cap 478, main spring 477, regulator body 464, piston seal 475, sealing ring 468, sealing ball 466, sealing ball spring 470, piercing lance 450, and piston 474.

Lance 450 is press-fit into the upstream end of valve chamber 465 and punctures compressed gas cartridge seal, distally located on neck of compressed gas cartridge when the same is brought into contact with lance 450. A lance as disclosed herein may be of any design that can pierce the seal of a compressed gas cartridge and allows release of compressed gas from the cartridge into the pressure-temperature regulator assembly in a manner that ensures proper operation of the transdermal delivery device. Such a lance design includes, e.g., a hollow piercing lance design and solid piercing lance design. Hollow piercing lance 450 is illustrated showing fluid port 452 disposed directly through the middle of piercing lance 450.

Formed within the interior wall of lance housing 444 is annular groove 451 that receives piercing lance sealing ring 458. Upon harnessing compressed gas cartridge sealing ring 448 creates an airtight seal between lance fluid port 452 and distal face of the neck of a compressed gas cartridge, e.g., compressed gas cartridge 312 shown in FIG. 3. Lance housing 444 currently has two major variations in the art being non-threaded and threaded. This embodiment illustrates non-threaded lance housing 444 and requires the use of a cartridge-retaining container to harness a compressed gas cartridge, see, e.g., cartridge-retaining container 326 and compressed gas cartridge 312, shown in FIG. 3.

Further downstream from piercing lance 450 is valve chamber 465. At the upper end of valve chamber 465 is valve assembly 452 that controls the flow of gas passing through pressure regulator 442. Main valve assembly 443 includes rigid valve ball 466, spring 470, and valve ball sealing ring 468. Rigid valve ball 466 is preferably made of a hard, metallic material such as stainless steel or hard-chrome plated steel. Other materials, even non-metallic, such as glass reinforced nylon possessing adequate material properties can also be used. Main valve assembly 443 is incorporated into body 464 in the following manner. Valve ball sealing ring 468 is inserted into valve chamber 465 and positioned within groove 461 provided at the downstream end of valve chamber 465. Following insertion of sealing ring 468, valve ball 466 is positioned in contact with sealing ring 468. The leading end coil of compression spring 470 is then positioned about the circumference of valve ball 466 and is compressed within valve chamber 465 by press-fitting piercing lance 450 into the upstream end of valve chamber 465.

Referring to FIG. 4, valve ball seat 469 extends into valve chamber 465 to limit the motion of valve ball 466 during inoperative periods and high-pressure situations such that sealing ring 468 is prevented from over-deformation and permanent deformation by rigid ball seat 469 that supports valve ball 466 when main valve assembly 443 is closed, thereby enabling long-term containment of unused gas. Additionally, this design of supportive valve ball seat enables extremely high pressures and pressure shocks to be reliably contained within valve chamber 465 as is the case upon lancing a compressed gas cartridge where initial cartridge lancing can slam main valve assembly 443 with high pressure gas. Additional benefits of rigid valve ball seat 469 limiting travel of valve ball 466 allows this valve assembly to handle cold and hot temperatures as well as temperature swings during service thereby affecting seal hardness as is common when harnessing high-pressure compressed gas cartridges, particularly at high flow rates where the gas is cool as it is changing from a substantially liquid phase in the cartridge to a gaseous phase as it is leaving the cartridge. The controlled limited compression of sealing ring 468 prevents sealing ring from taking a permanent compression set yet allows for a reliable seal.

Immediately downstream from valve ball seat 469 is a plunger channel 473. Plunger channel 473 is dimensioned to receive a plunger 472 that communicates at a contact interface 467 with valve ball 466 to open valve assembly 443. The dimensions of plunger 472 are slightly smaller than plunger channel 473. Two reasons for these dimensions are to allow plunger 472 to freely move in plunger channel 473 as well as allowing means for a fluid connection between valve chamber 465 and downstream to a regulated pressure contained on the bottom side of a piston 474 as will be discussed next.

Plunger 472 extends from plunger to valve ball interface 467, downstream through plunger channel 473 and integrally connects to piston 474. In this exemplary embodiment, plunger 472 is monolithically formed as a feature of piston 474. Piston guide 484 is formed as an integral feature of regulator body 464 and is dimensioned slightly smaller than piston skirt inside diameter thereby preventing an interference fit. These stated dimensions allow piston 474 to freely move along guide 484 as well as allowing means for fluid passage between plunger channel 473 and a piston bore 480, also formed as an integral part of regulator body 464.

In use, the pressure contained in piston bore 480 on the (bottom) plunger side of piston 474 will be defined as regulated pressure herein expressed as σ₂. Piston 474 freely moves in piston bore 480 aligned by guide 484, and isolates regulated pressure σ₂ from the topside of piston 474 by piston seal 475.

Located on the topside of piston 474 is a compression piston spring 477. Piston spring 477 is inserted through the top of regulator body 464, contacting the top of piston 474 and retained by a cap 478. Cap 478 comprises a female thread at 487 and correspondingly threads to a male thread at 489 onto integrated threads in regulator body 464. Cap 478 has grip features molded into the outer diameter enabling an easy grip when adjusting preload on piston spring 477. Additionally, cap 478 has a large hole 498 in its top that allows a hose (not shown) to be mechanically connected to piston 474 and pass out of regulator assembly 442. Large hole 498 also allows any pressure on the topside of piston 474 to vent to the atmosphere.

Prior to piston 474 bottoming out on a travel limit shelf 481 in piston bore 480, plunger 472 contacts valve ball 466 at plunger to valve ball interface 467 and opens valve assembly 443. When valve assembly 443 is open, pressure equilibrium is achieved between lance fluid port 452 which is in pressure equilibrium with a compressed gas cartridge as disclosed herein, through valve chamber 465, all the way downstream to piston bore 480, contained by the bottom (plunger side) of piston 474 by piston seal 475. When no compressed gas cartridge is attached to regulator 442, valve 443 is biased in the open position by the force of piston spring 477.

Upon introduction of a high-pressure fluid from lancing a compressed gas cartridge, that exceeds 800 pounds per square inch pressure at room temperature for carbon dioxide, this fluid travels through valve assembly 443 and creates a new regulated pressure σ₂, pushing up on piston 474 and piston spring 477. The selected spring rate of piston spring 477 combined with the pre-loading of piston spring 477 by cap 498 determines regulated pressure σ₂. A higher spring force creates a higher regulated pressure σ₂.

Exit conduit 482 of regulated pressure σ₂ taps off the top of piston 474. Alternate exit conduit 493 of regulated fluid pressure could tap into regulator body 464 anywhere downstream from valve assembly 443 within pressurized piston bore 480 contained by piston seal 475 such as through a port in regulator body 464 rather than through the top of piston 474. Conduit is typical hose barb, NPT (National Pipe) threads, or similar connection and leads to any pneumatic or hydraulic device requiring a regulated, substantially constant working pressure to operate.

As regulated pressure σ₂ is tapped off exit conduit 482, regulated pressure σ₂ decreases, and in effect reduces the pressure contained on the bottom side of piston 474, allowing piston 474 to move down in piston bore 480 ultimately opening valve assembly 443 with plunger 472. Opened valve assembly 443 again introduces additional high-pressure fluid through plunger channel 473 and increases the pressure contained by piston 474, in effect, biasing piston 474 upward in piston bore 480 closing valve assembly 443, thereby substantially maintaining a consistent regulated pressure σ₂.

Over-pressurization prevention feature 490 is illustrated in FIG. 4 more specifically comprising a negative vent or plurality of negative vents 492.

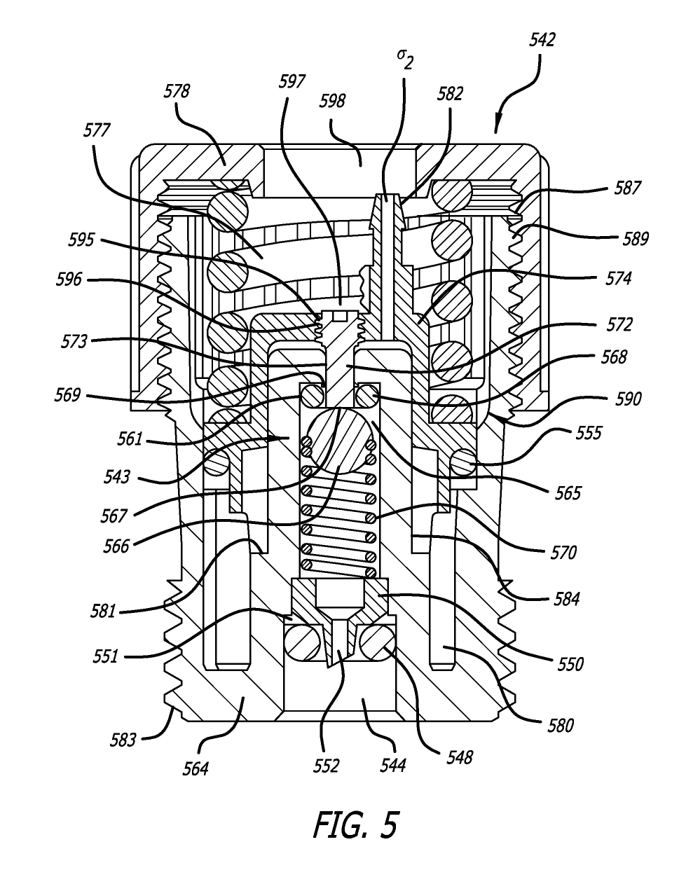

In another embodiment, as shown in FIG. 5, pressure-temperature regulator assembly 542 further comprising threaded plunger 595 that allows for the height of the plunger 572 to be adjusted for added tuning capabilities. During operation, threaded plunger 595 mates with a piston female thread 596. Slot 597 located on the top of threaded plunger 595 allows an operator to thread plunger 595 higher or lower into piston 574. The purpose of the adjustable plunger height allows the ability for one to tune the regulator to blow off at a desired back-pressure, independent of preload on piston spring 577. In operation, cap 578 preloads piston spring 577 thus providing a substantially constant spring force on regulator piston 574. Allowing plunger 572 to be moveable with respect to piston 574, the relationship between piston equilibrium position (and position of piston seal 575) and opening degree of valve assembly 543 can be tailored. Mostly to benefit from this feature is that the blow-off pressure is tunable. Rather than make bore height h (FIG. 5) of regulator body over-pressurization prevention feature 590 differ in order to achieve vents at differing bore heights h, one species of regulator body 564 comprising negative vent(s) 592 in the same location can be used with tunable piston 574 and plunger 572 to achieve desired blow-off pressures rather than produce a variety of different regulator bodies 564 possessing differing bore height h.

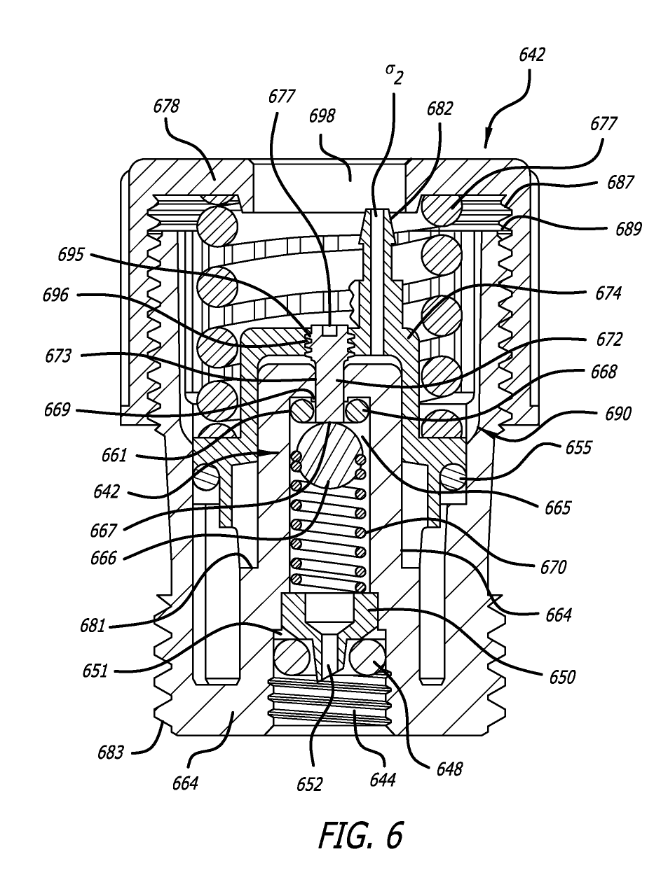

In yet another embodiment, as shown in FIG. 6, pressure-temperature regulator assembly 642 comprising the capability to dispense compressed gas cartridges possessing a threaded neck without the aid of a cartridge-retaining container disclosed herein. An additional feature to regulator body 664 differs slightly from regulator body 464 (FIG. 4) in that lance housing 644 is internally threaded. A threaded lance housing allows the use of a threaded compressed gas cartridge to harness the cartridge to the pressure-temperature regulator assembly. As such, a cartridge retaining container as disclosed herein is not necessary to harness the compressed gas cartridge to the pressure-temperature regulator assembly. A compressed gas cartridge comprising a threaded neck is not illustrated in the FIGS. Similarly, non-threaded neck compressed gas cartridge utilized in conjunction with cartridge-retaining container can still be dispensed with regulator body 664. If a non-threaded compressed gas cartridge is to be dispensed, a cartridge-retaining container as disclosed herein is required to engage the compressed gas cartridge to pressure regulator 642. Piston 674 and adjustable height plunger 672 share the same user-tunable blow-off pressure benefits as described in the embodiment illustrated and described in FIG. 5.

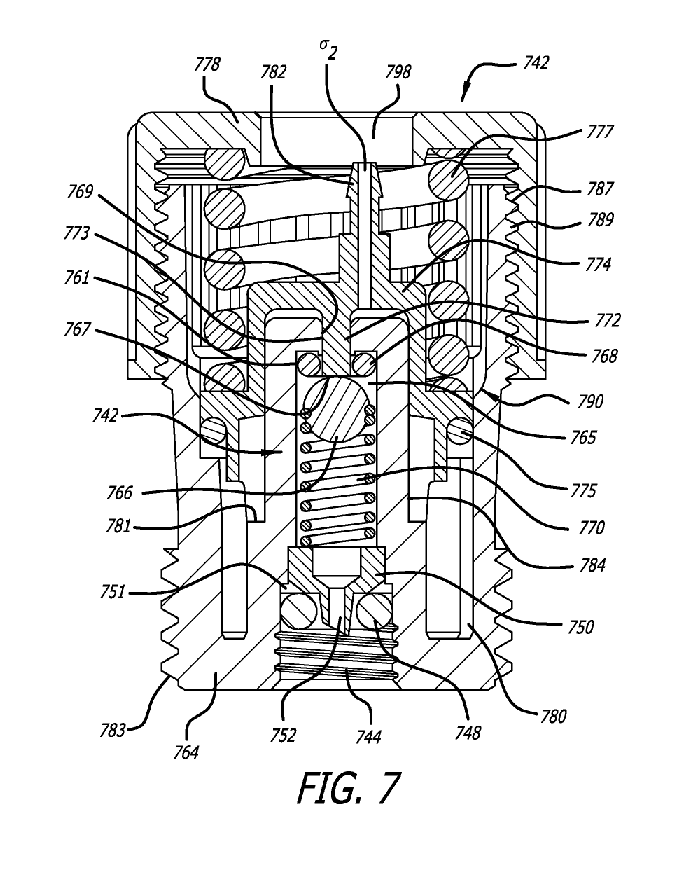

In yet another embodiment, as shown in FIG. 7, pressure-temperature regulator assembly 742 comprises regulator body 764 including an internally threaded lance housing 744 capable of threadably mating to a threaded neck compressed gas cartridge (cartridge not illustrated). Regulator 742 features the same type of piston 774 and plunger 772 as exemplified in the embodiment illustrated and described in FIG. 4.

A pressure-temperature regulator assembly is adjusted to dispense a gas for an appropriate amount of time to ensure an appropriate amount of gas is dissolvent into the liquid. In one embodiment, the pressure-temperature regulator assembly is adjusted to dispense a gas for, e.g., about 3 minutes, about 5 minutes, about 7 minutes, about 10 minutes, about 12 minutes, about 15 minutes, about 18 minutes, or about 20 minutes. In another embodiment, the pressure-temperature regulator assembly is adjusted to dispense a gas for, e.g., at least 3 minutes, at least 5 minutes, at least 7 minutes, at least 10 minutes, at least 12 minutes, at least 15 minutes, at least 18 minutes, or at least 20 minutes. In yet another embodiment, the pressure-temperature regulator assembly is adjusted to dispense a gas for, e.g., about 3 minutes to about 5 minutes, about 3 minutes to about 10 minutes, about 3 minutes to about 15 minutes, about 3 minutes to about 20 minutes, about 5 minutes to about 10 minutes, about 5 minutes to about 15 minutes, or about 5 minutes to about 20 minutes.

A control switch assembly disclosed herein comprises comprising an actuator, a switch body, an inlet port and an outlet port and may operate by a mechanical or an electronic design. A control switch assembly disclosed herein is designed to control when low pressure gas leaving the regulator assembly is allowed to enter into the fluid chamber assembly.

In one embodiment, as shown in FIG. 8, control switch assembly 844 comprises actuator 850, switch body 852, inlet port 854, flow ball seat valve 856, and flow valve insert 858. Inlet port 854 is in communication with an outlet port of a pressure-temperature regulator assembly disclosed herein. However, actuator 850 prevents passage of the gas into flow ball seat valve 856. Upon activation of actuator 850, a channel is formed that establishes communication between inlet port 854 and flow ball seat valve 856, thereby enabling gas to enter into flow ball seat valve 856. Flow ball seat valve 856 comprises ball seat valve body 858 housing seat valve ball 860, ball spring 862, and ball seat valve outlet port 864. Activation of actuator 850 releases tension in ball spring 862 which reduces pressure on ball 860 forced against O-ring 866 by the tension of ball spring 862. With pressure removed, gas can flow through the flow ball seat valve 856, exiting via ball seat valve outlet port 864. A channel in communication with control switch assembly 844 and a fluid chamber assembly disclosed herein allows the gas to flow into the fluid chamber assembly. This communication channel is formed by an inlet port a fluid chamber assembly as disclosed herein and flow valve insert 858.

A fluid chamber assembly disclosed herein comprises a fluid container, and inlet port and an outlet port. The fluid container holds the liquid that will be supersaturated by the gas entering into the chamber from a pressure-temperature regulator assembly. The liquid can be water, a physiologically buffered solution, or any other suitable liquid. A suitable liquid is one that 1) allows for an appropriate amount of gas to be dissolved into the liquid in order to produce a vapor comprising liquid particles including a supersaturated amount of a therapeutic agent; and 2) maintains, enables, or ensures the activity of a therapeutic agent, thereby ensuring that a therapeutically effective amount of the agent is received by an individual upon administration. For example, carbon dioxide exits in a gaseous form and a molecular form. It is the molecular form of carbon dioxide that is capable of dissolving in a liquid, such as, e.g., water, which allows for the easily absorbed of carbon dioxide through the skin. Conversely, at higher pH, carbon dioxide tends to change to carbonic acid (H₂CO₃) and bicarbonate ions which are not easily absorbed through the skin. The lower the pH of the liquid, the more molecular carbon dioxide exists. As such, when the gas is carbon dioxide, the pH of the liquid should be slightly acidic, such as, e.g., no more then about pH 6, no more then about pH 5.5, no more then about pH 5, no more then about pH 4.5, or no more then about pH 4.

Alternatively, another substance capable of dissolving a supersaturated amount of gas may be used instead of a liquid. Non-limiting examples of such a substance include colloids, such as, e.g., foams, liquid aerosols, emulsions, gels, and sols.

A liquid disclosed herein comprises a therapeutic agent. As used herein, the term “therapeutic agent” is synonymous with “active ingredient” and refers to used to any substance that provides a beneficial effect to an individual being administered the therapeutic agent.

One type of therapeutic agent is the gas that has been dissolved into the liquid as disclosed herein. An exemplary gas that is a therapeutic agent is carbon dioxide. Current uses of gases in medicine are rapidly being explored because these molecules are important biological messengers. For example, increasing the level of carbon dioxide in the blood decreases the pH due to the conversion of carbon dioxide into bicarbonate. This decreased pH enables oxygen to more readily dissociate from hemoglobin, referred to as the “Bohr effect.” Additionally, an increased level of carbon dioxide improves circulation and blood flow by triggering the release vasodilatory agents which dilate blood vessels in an effort to increase oxygen supply. As such, increasing carbon dioxide level increases tissue oxygen which, in turn, increases dilation of blood vessels which allows for the delivery of more nutrients to cells, and increasing higher oxygen supply to cells thereby enhancing cellular metabolism. As such, increasing the level of tissue oxygen in this manner provides many beneficial effects that promote skin health including, without limitation, promoting wound healing, improving skin texture, and providing anti-aging effects.

Another type of therapeutic agent that can be administered by a transdermal delivery device disclosed herein is a drug that can either be dissolved in a liquid disclosed herein or become part of the vapor upon vaporization. Approximately half of the pharmaceutical drugs available on the market today possess a molecular affinity for water. This affinity manifests itself in a tendency to dissolve in, mix with, or absorb water. Therapeutic agents with these characteristics are referred to as hydrophilic therapeutic agents and comprise small molecule or chemical drugs as well a biologics. Hydrophilic therapeutic agents include, without limitation, nicotine antihistamines, β-blockers, calcium channel blockers, non-steroidal anti-inflammatory drugs, contraceptives, anti-arrhythmic drugs, insulin, antivirals, hormones, α-interferon, and chemotherapeutic agents.

Another type of therapeutic agent that can be administered by a transdermal delivery device disclosed herein is a vitamin that can either be dissolved in a liquid disclosed herein or become part of the liquid particle upon vaporization.

In one embodiment, the amount of gas dissolved in the liquid is, e.g., about 30,000 ppm, about 35,000 ppm, about 40,000 ppm, about 45,000 ppm, about 50,000 ppm, about 55,000 ppm, or about 60,000 ppm. In another embodiment, the amount of gas dissolved in the liquid is, e.g., at least 30,000 ppm, at least 35,000 ppm, at least 40,000 ppm, at least 45,000 ppm, at least 50,000 ppm, at least 55,000 ppm, or at least 60,000 ppm. In yet another embodiment, the amount of gas dissolved in the liquid is, e.g., at most 30,000 ppm, at most 35,000 ppm, at most 40,000 ppm, at most 45,000 ppm, at most 50,000 ppm, at most 55,000 ppm, or at most 60,000 ppm. In still another embodiment, the amount of gas dissolved in the liquid is between, e.g., about 30,000 ppm to about 35,000 ppm, about 30,000 ppm to about 40,000 ppm, about 30,000 ppm to about 45,000 ppm, about 30,000 ppm to about 50,000 ppm, about 35,000 ppm to about 40,000 ppm, about 35,000 ppm to about 45,000 ppm, about 35,000 ppm to about 50,000 ppm, about 40,000 ppm to about 45,000 ppm, about 40,000 ppm to about 50,000 ppm, or about 50,000 ppm to about 60,000 ppm.

In an embodiment where the therapeutic agent is not the dissolved gas, the agent is contained in the liquid placed in the fluid container. Additionally, a liquid placed into the fluid container may comprise both the additional therapeutic agent as well as a dissolved gas that also provides a therapeutic effect.

A fluid chamber assembly may optionally comprise a fluid container cap that detachably engages a fluid container disclosed herein. The ability to detach a fluid container as disclosed herein allows for the refilling of a liquid as needed. For example, in an application involving the treatment of a wound, the liquid may contain both a wound healing drug like cyclosporine as well as dissolved molecular carbon dioxide.

An inlet port as disclosed herein is designed to receive the low pressure gas flowing from the pressure-temperature regulator assembly and channels the gas into the fluid chamber assembly. Once in the fluid chamber assembly, the gas will dissolve into the liquid contained in the fluid container to produce a liquid comprising a supersaturated amount of dissolved gas molecules. As used herein, the term “supersaturated” when used in reference to “supersaturated amount of dissolved gas molecules” refers to a liquid disclosed herein that contains more of a dissolved gas than the liquid can accommodate under ambient temperature and air pressure, typically measured at 25° C. and 1 atm. For example, with reference to a transdermal delivery device disclosed herein, the pressure of dissolved gas in the fluid chamber assembly is greater than the pressure of the gas outside the assembly. In one embodiment, an inlet port as disclosed herein comprises a check value, a spring and a poppet.

An outlet port as disclosed herein is designed to release a vapor including a supersaturated amount of dissolved gas molecules and/or a therapeutic agent at ambient pressure from the fluid chamber assembly into an open-ended delivery outlet where it can be administered to an individual. In one embodiment, an outlet port as disclosed herein comprises a check value, a spring and a poppet. Vaporization of the liquid comprising a supersaturated amount of dissolved gas is achieved when the pressure inside the liquid container is sufficient to expel the liquid through the outlet port. In aspects of this embodiment, vaporization of the liquid comprising a supersaturated amount of dissolved gas is achieved when the pressure inside the liquid container is, e.g., about 15 psi, about 20 psi, about 25 psi, about 30 psi, about 35 psi, about 40 psi, about 45 psi, or about 50 psi. In other aspects of this embodiment, vaporization of the liquid comprising a supersaturated amount of dissolved gas is achieved when the pressure inside the liquid container is, e.g., at least 15 psi, at least 20 psi, at least 25 psi, at least 30 psi, at least 35 psi, at least 40 psi, at least 45 psi, or at least 50 psi. In yet other aspects of this embodiment, vaporization of the liquid comprising a supersaturated amount of dissolved gas is achieved when the pressure inside the liquid container is, e.g., at most 15 psi, at most 20 psi, at most 25 psi, at most 30 psi, at most 35 psi, at most 40 psi, at most 45 psi, or at most 50 psi. In still other aspects of this embodiment, vaporization of the liquid comprising a supersaturated amount of dissolved gas is achieved when the pressure inside the liquid container is from, e.g., about 15 psi to about 50 psi, about 20 psi to about 50 psi, about 25 psi to about 50 psi, about 30 psi to about 50 psi, about 35 psi to about 50 psi, about 15 psi to about 45 psi, about 20 psi to about 45 psi, about 25 psi to about 45 psi, about 30 psi to about 45 psi, about 35 psi to about 45 psi, about 15 psi to about 40 psi, about 20 psi to about 40 psi, about 25 psi to about 40 psi, about 30 psi to about 40 psi, about 15 psi to about 35 psi, about 20 psi to about 35 psi, about 25 psi to about 35 psi, about 15 psi to about 30 psi, or about 20 psi to about 30 psi.

A vapor as disclosed herein comprises liquid particles and a supersaturated amount of dissolved gas molecules. A vapor can be a solution comprising a liquid and a gas, or a liquid aerosol, which is a colloid composition comprising a liquid and a gas. When the therapeutic agent is not the dissolved gas, a vapor also comprises a therapeutic agent as disclosed herein.

Vaporization creates liquid particle of an average size small enough to be able to enter the pores of the skin. In one embodiment, the average size of a liquid particle is, e.g., about 100 μm, about 75 μm, about 50 μm, or about 25 μm. In another embodiment, the average size of a liquid particle is, e.g., no more than 100 μm, no more than 75 μm, no more than 50 μm, or no more than 25 μm. In yet another embodiment, the average size of a liquid particle is, e.g., about 1 μm to about 100 μm, about 1 μm to about 75 μm, about 1 μm to about 50 μm, about 1 μm to about 25 μm, about 5 μm to about 100 μm, about 5 μm to about 75 μm, about 5 μm to about 50 μm, about 5 μm to about 25 μm, about 10 μm to about 100 μm, about 10 μm to about 75 μm, about 10 μm to about 50 μm, or about 10 μm to about 25 μm.

A fluid chamber assembly may optionally comprise a pressure relief valve as a safety measure for avoiding an over-pressurization of the vapor producing assembly or component thereof. In one embodiment, a pressure relief valve is, e.g., a 30 psi valve, a 35 psi valve, a 40 psi valve, a 45 psi valve, or a 50 psi valve.

A fluid chamber assembly may optionally comprise a baffle assembly comprising one or more conical baffles or mixing elements. The baffle assembly is connected to the fluid container or fluid container cap. The baffles are positioned in a column configuration with each baffle above and partially overlapping the other and their circular base sides face away from the inlet port. Narrow connecting pieces at the periphery of this column position the baffles in place. As low pressure gas enters into the fluid container via the inlet port, the gas flows pass over the baffles to enhance the mixing of the gas and liquid. As such, the baffles are designed to speed up and/or increase the amount of gas dissolved into the liquid. In one embodiment, fluid chamber assembly does not comprise a baffle assembly.

In one embodiment, as shown in FIG. 9, fluid chamber assembly 946 comprises fluid container 950, fluid container cap 952 containing inlet port 954 including inlet poppet 956, inlet spring 958 and inlet check value 960, and outlet port 962 including inlet poppet 964, inlet spring 966 and inlet check value 968. A liquid as disclosed herein is placed into fluid container 950 and attached to fluid container cap 952 via threads. Gas enters fluid chamber assembly 946 via inlet port 954 where the gas dissolves into the liquid. After a predetermined period of time, the liquid comprising a supersaturated amount of gas dissolved gas is released via outlet port 962 as a vapor.

Aspects of the present specification disclose, in part, a method of producing a substance comprising a supersaturated amount of dissolved gas. As used herein, the term “substance” includes any material capable of dissolving a supersaturated amount of gas. Non-limiting examples of a substance include liquids and colloids, such as, e.g., foams, liquid aerosols, emulsions, gels, and sols. In the method disclosed herein, a substance is placed in an air-tight container and the substance is then exposed to a gas. Upon such exposure, the gas dissolves into the substance in an amount greater than the substance could dissolve at 25° C. and 1 atm. The resulting substance supersaturated with the dissolved gas can then be administered to an individual to treat a condition as disclosed herein.

In one embodiment, the amount of gas dissolved in the substance is, e.g., about 30,000 ppm, about 35,000 ppm, about 40,000 ppm, about 45,000 ppm, about 50,000 ppm, about 55,000 ppm, or about 60,000 ppm. In another embodiment, the amount of gas dissolved in the substance is, e.g., at least 30,000 ppm, at least 35,000 ppm, at least 40,000 ppm, at least 45,000 ppm, at least 50,000 ppm, at least 55,000 ppm, or at least 60,000 ppm. In yet another embodiment, the amount of gas dissolved in the substance is, e.g., at most 30,000 ppm, at most 35,000 ppm, at most 40,000 ppm, at most 45,000 ppm, at most 50,000 ppm, at most 55,000 ppm, or at most 60,000 ppm. In still another embodiment, the amount of gas dissolved in the substance is between, e.g., about 30,000 ppm to about 35,000 ppm, about 30,000 ppm to about 40,000 ppm, about 30,000 ppm to about 45,000 ppm, about 30,000 ppm to about 50,000 ppm, about 35,000 ppm to about 40,000 ppm, about 35,000 ppm to about 45,000 ppm, about 35,000 ppm to about 50,000 ppm, about 40,000 ppm to about 45,000 ppm, about 40,000 ppm to about 50,000 ppm, or about 50,000 ppm to about 60,000 ppm.

In another embodiment, a method of producing a substance comprising a supersaturated amount of dissolved gas disclosed herein is performed using a transdermal delivery device disclosed herein. For example, a fluid chamber assembly can be filled with a liquid or a colloid as disclosed herein and the device activated to produce a liquid or a colloid comprising a supersaturated amount of dissolved gas.

Aspects of the present specification disclose, in part, a method of transdermally administering a therapeutically effective amount of therapeutic agent disclosed herein. In one aspect, the method disclosed herein comprises the step of administering a vapor comprising a supersaturated amount of dissolved gas to an individual using a transdermal delivery device disclosed herein. In another aspect, the method disclosed herein comprises the step of administering a vapor comprising a supersaturated amount of dissolved gas and a therapeutic agent to an individual using a transdermal delivery device disclosed herein. Administration of the gas and/or the therapeutic agent typically treats a symptom associate with a condition.

In another aspect, the method disclosed herein comprises the step of administering a liquid aerosol comprising a supersaturated amount of dissolved gas to an individual using a transdermal delivery device disclosed herein. In yet another aspect, the method disclosed herein comprises the step of administering a liquid aerosol comprising a supersaturated amount of dissolved gas and a therapeutic agent to an individual using a transdermal delivery device disclosed herein. Administration of the gas and/or the therapeutic agent typically treats a symptom associate with a condition.

In another aspect, the method disclosed herein comprises the step of administering a foam comprising a supersaturated amount of dissolved gas to an individual using a transdermal delivery device disclosed herein. In yet another aspect, the method disclosed herein comprises the step of administering a foam comprising a supersaturated amount of dissolved gas and a therapeutic agent to an individual using a transdermal delivery device disclosed herein. Administration of the gas and/or the therapeutic agent typically treats a symptom associate with a condition.

In another aspect, the method disclosed herein comprises the step of administering an emulsion comprising a supersaturated amount of dissolved gas to an individual using a transdermal delivery device disclosed herein. In yet another aspect, the method disclosed herein comprises the step of administering an emulsion comprising a supersaturated amount of dissolved gas and a therapeutic agent to an individual using a transdermal delivery device disclosed herein. Administration of the gas and/or the therapeutic agent typically treats a symptom associate with a condition.

In another aspect, the method disclosed herein comprises the step of administering a gel comprising a supersaturated amount of dissolved gas to an individual using a transdermal delivery device disclosed herein. In yet another aspect, the method disclosed herein comprises the step of administering a gel comprising a supersaturated amount of dissolved gas and a therapeutic agent to an individual using a transdermal delivery device disclosed herein. Administration of the gas and/or the therapeutic agent typically treats a symptom associate with a condition.

In another aspect, the method disclosed herein comprises the step of administering a sol comprising a supersaturated amount of dissolved gas to an individual using a transdermal delivery device disclosed herein. In yet another aspect, the method disclosed herein comprises the step of administering a sol comprising a supersaturated amount of dissolved gas and a therapeutic agent to an individual using a transdermal delivery device disclosed herein. Administration of the gas and/or the therapeutic agent typically treats a symptom associate with a condition.

Aspects of the present specification disclose, in part, a method of treating a condition of an individual. A method of treating a condition disclosed herein comprises the step of administering a composition comprising a substance including a supersaturated amount of dissolved gas and a therapeutic agent using a transdermal delivery device as disclosed herein to a body part of the individual suffering from a condition, wherein the administration of the composition reduces a symptom associated with condition. A substance may be a liquid aerosol, foam, emulsion, gel, sol, or other substance that can become supersaturated with an amount of dissolved gas. A condition includes an imperfection, a defect, a disease, and/or a disorder for which relief is sought by the individual suffering from the condition. A therapeutic agent is transdermally administered to an individual. An individual is typically a mammal and this term includes a human being. As such, the transdermal delivery device is useful for cosmetic, medical and veterinarian applications.

In one embodiment, a method of treating a condition of an individual comprises the step of administering a composition comprising a vapor including a supersaturated amount of dissolved gas with or without another therapeutic agent using a transdermal delivery device as disclosed herein to a body part of the individual suffering from a condition, wherein the administration of the composition reduces a symptom associated with condition. In an aspect of this embodiment, the dissolved gas is carbon dioxide. In another aspect of this embodiment, the dissolved gas is carbon dioxide, which also serves as the therapeutic agent.

In one embodiment, a method of treating a condition of an individual comprises the step of administering a composition comprising a liquid aerosol including a supersaturated amount of dissolved gas with or without another therapeutic agent using a transdermal delivery device as disclosed herein to a body part of the individual suffering from a condition, wherein the administration of the composition reduces a symptom associated with condition. In an aspect of this embodiment, the dissolved gas is carbon dioxide. In another aspect of this embodiment, the dissolved gas is carbon dioxide, which also serves as the therapeutic agent.

As used herein, the term “treating,” refers to reducing or eliminating in an individual a cosmetic or clinical symptom associated with a condition; or delaying or preventing in an individual the onset of a cosmetic or clinical symptom associated with a condition. For example, the term “treating” can mean reducing a symptom associated with a condition by, e.g., at least 20%, at least 30%, at least 40%, at least 50%, at least 60%, at least 70%, at least 80%, at least 90% or at least 100%. The effectiveness of a therapeutic agent disclosed herein in treating a condition can be determined by observing one or more cosmetic, clinical symptoms, and/or physiological indicators associated with the condition. An improvement in a condition also can be indicated by a reduced need for a concurrent therapy. Those of skill in the art will know the appropriate symptoms or indicators associated with specific condition and will know how to determine if an individual is a candidate for treatment with a therapeutic agent by using the transdermal delivery device disclosed herein.

Aspects of the present specification provide, in part, administering a therapeutically effective amount of a therapeutic agent disclosed herein. As used herein, the term “therapeutically effective amount” is synonymous with “therapeutically effective dose” and refers to the minimum dose of therapeutic agent disclosed herein necessary to achieve the desired therapeutic effect and includes a dose sufficient to reduce a symptom associated with a condition. In aspects of this embodiment, a therapeutically effective amount of a therapeutic agent disclosed herein reduces a symptom associated with a condition by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a therapeutic agent disclosed herein reduces a symptom associated with a condition by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a therapeutic agent disclosed herein reduces a symptom associated with a condition by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other aspects of this embodiment, a therapeutically effective amount of a therapeutic agent disclosed herein is the dosage sufficient to reduces a symptom associated with a condition for, e.g., at least one week, at least one month, at least two months, at least three months, at least four months, at least five months, at least six months, at least seven months, at least eight months, at least nine months, at least ten months, at least eleven months, or at least twelve months.

The actual therapeutically effective amount of a therapeutic agent disclosed herein to be administered to an individual can be determined by a person of ordinary skill in the art by taking into account factors, including, without limitation, the type of condition, the location of the condition, the cause of the condition, the severity of the condition, the duration of treatment, the degree of relief desired, the duration of relief desired, the particular therapeutic agent used, the rate of excretion of the therapeutic agent used, the pharmacodynamics of the therapeutic agent used, the nature of the other compounds to be included in the vapor, the particular characteristics, history and risk factors of the individual, such as, e.g., age, weight, general health and the like, the response of the individual to the treatment, or any combination thereof. A therapeutically effective amount of a therapeutic agent disclosed herein can thus readily be determined by the person of ordinary skill in the art considering all criteria and utilizing his best judgment on the individual's behalf.

As a non-limiting example, a vapor comprising water particles including a supersaturated amount of dissolved molecular carbon dioxide can be administered using the transdermal delivery device disclosed herein to treat an individual with limb ischemia. Such a treatment can improve blood circulation and oxygenation of the limb, thereby treating the ischemic limb.

As another non-limiting example, a vapor comprising water particles including a supersaturated amount of dissolved molecular carbon dioxide can be administered using the transdermal delivery device disclosed herein to treat an individual with pale skin. Such a treatment can improve blood circulation and oxygenation of the skin, thereby treating the pale skin.

As yet another non-limiting example, a vapor comprising water particles including a supersaturated amount of dissolved molecular carbon dioxide can be administered using the transdermal delivery device disclosed herein to treat an individual with a soft tissue condition. Such a treatment can improve blood circulation and oxygenation of the area comprising the soft tissue condition, thereby treating the soft tissue condition. Non-limiting examples of a soft tissue condition include a facial imperfection, defect, disease or disorder, such as, e.g., dermal divots, sunken checks, thin lips, nasal imperfections or defects, retro-orbital imperfections or defects, a facial fold, line and/or wrinkle like a glabellar line, a nasolabial line, a perioral line, and/or a marionette line, and/or other contour deformities or imperfections of the face.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of excludes any element, step, or ingredient not specified in the claims. The transition term “consisting essentially of limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1-15. (canceled)

16. A transdermal delivery device comprising:
a) a housing including
i) an external body shell comprising a fluid chamber assembly access cover detachably engaged with the external body shell;
ii) an interior compartment including an open-ended delivery outlet and a vapor producing assembly compartment; and
iii) a cartridge retaining container comprising an external covering shell and an internal cartridge compartment configured to hold a compressed gas cartridge, wherein the cartridge retaining container is detachably engaged with the external body shell;
wherein the vapor producing assembly compartment is located between the open-ended delivery outlet and the cartridge retaining container; and
b) a vapor producing assembly including
i) a fluid chamber assembly comprising a fluid container and a removable fluid container cap; and
ii) a pressure-temperature regulator assembly comprising at least two pressure regulators connected via an adaptor, wherein the pressure-temperature regulator assembly is set to reduce the pressure and increase the temperature of a compressed gas from the compressed gas cartridge;
wherein the vapor producing assembly is substantially contained within the vapor producing assembly compartment.

17. The transdermal delivery device of claim 16, wherein the external covering shell defines the internal cartridge compartment.

18. The transdermal delivery device of claim 16, wherein the device further includes a control switch assembly.

19. The transdermal delivery device of claim 18, wherein the control switch assembly is a mechanical switch or an electronic switch.

20. The transdermal delivery device of claim 16, wherein the compressed gas cartridge is a 16 g compressed gas cartridge.

21. The transdermal delivery device of claim 16, wherein the compressed gas cartridge includes a compressed gas.

22. The transdermal delivery device of claim 21, wherein the compressed gas is carbon dioxide.

23. The transdermal delivery device of claim 21, wherein the pressure-temperature regulator assembly is configured to reduce a pressure of the compressed gas to below 40 psi.

24. The transdermal delivery device of claim 21, wherein the pressure-temperature regulator assembly is configured to increase a temperature of the compressed gas to at least 0° C.

25. The transdermal delivery device of claim 16, wherein the fluid container includes water.

26. A method of transdermally administering a therapeutically effective amount of dissolved molecular carbon dioxide comprising the step of administering a composition comprising a vapor including a supersaturated amount of dissolved molecular carbon dioxide to an individual using a transdermal delivery device according to claim 1.

27. The method of claim 26, wherein the amount of dissolved molecular carbon dioxide is at least 30,000 ppm.

28. The method of claim 26, wherein a fluid in the fluid container is water at pH 4.

29. A method of transdermally administering a therapeutically effective amount of a therapeutic agent comprising the step of administering a composition comprising a vapor including a supersaturated amount of dissolved molecular carbon dioxide and a therapeutic agent to an individual using a transdermal delivery device according to claim 1.

30. The method of claim 29, wherein the amount of dissolved molecular carbon dioxide is at least 30,000 ppm.

31. A method of producing a substance comprising a supersaturated amount of dissolved gas, the method comprising the steps of:
a) placing the substance in an air-tight container; and
b) exposing the substance to carbon dioxide, wherein upon exposure, the carbon dioxide dissolves into the substance in an amount greater than the substance could dissolve at 25° C. and 1 atm.

32. The method of claim 31, wherein the substance is a liquid or a colloid.

33. The method of claim 32, wherein the colloid is a foam, a liquid, an aerosol, an emulsion, a gel, or a sol.

34. The method of claim 33, wherein a liquid is water at pH 4.

35. The method of claim 31, wherein dissolved carbon dioxide is produced at a concentration of at least 30,000 ppm.
